(19) United States
(12) Patent Application Publication
(10) Pub. No.: US 2025/0128263 A1
(43) Pub. Date: Apr. 24, 2025

(54) MOVING DEVICE AND CONTROL METHOD THEREOF FOR NUCLEIC ACID EXTRACTION SYSTEM

(71) Applicant: ARISE BIOTECH CORPORATION, New Taipei City (TW)

(72) Inventors: Chung-Che LO, New Taipei City (TW); Shan-Yi YEN, New Taipei City (TW); Yi-Chi WANG, New Taipei City (TW); Nien-Ting CHEN, New Taipei City (TW); Chih-Wei LAI, New Taipei City (TW)

(21) Appl. No.: 18/606,267

(22) Filed: Mar. 15, 2024

(30) Foreign Application Priority Data
Oct. 20, 2023 (TW) 112140271

(51) Int. Cl. B01L 9/02; B01L 3/00; B01L 9/06; C12N 15/10

(52) U.S. Cl. CPC B01L 9/02; B01L 3/5021; B01L 3/50825; B01L 9/06; C12N 15/1017; B01L 2200/025; B01L 2200/18

(57) ABSTRACT

A moving device, applied in a nucleic acid extraction system and cooperated with a plurality of microtube components, the moving device includes: a workbench; a moving component, disposed on the workbench; a control element, disposed on the moving component and electrically connected to the moving component; a first motor, disposed on the moving component and electrically connected to the control element; a first rotating rod, connected to the first motor; a second motor, disposed on the moving component and electrically connected to the control element; a second rotating rod, connected to the second motor; and a telescopic component, disposed on the moving component corresponding to the first rotating rod and the second rotating rod, electrically connected to the control element, and movable between a first position and a second position.

### BACKGROUND

### Technical Field

The present disclosure relates to a moving device and a control method, particularly relates to a moving device and a control method applied in a nucleic acid extraction system.

### Description of Related Art

Nucleic acid is one of the most basic substances of life and plays an important decisive role in the growth, heredity, mutation, and other phenomena of organisms. Therefore, the current society is still continuously investing in research on the structure, function, and application of DNA, RNA, and such nucleic acid molecules, especially on the application of nucleic acid molecules in the diagnosis and treatment of diseases and genetic engineering.

Generally, in spin column-based nucleic acid purification, first, biological samples are first manually placed into the column tube of the microcontainer. Then the microcontainer is manually placed in a centrifuge for centrifugation. Then the column tube containing the nucleic acid is manually taken out. Then the collection tube containing the waste liquid is manually taken out. The above all require a lot of manpower and time.

In view of this, how to carry out the process of purifying nucleic acids by the column tube method in an automated manner is actually one of the current problems that need to be solved urgently.

### SUMMARY

The present disclosure provides a moving device and control method thereof for a nucleic acid extraction system. The present disclosure may convert the related-art that manually tightens the sealing screw cover, takes the microtube component, moves the microtube component, and removes the microtube component and other cumbersome, repetitive, and boring processes to be automated to save user's labor.

The present disclosure provides a moving device, applied in a nucleic acid extraction system and cooperated with a plurality of microtube components, the moving device including: a workbench; a moving component, disposed on the workbench; a control element, disposed on the moving component and electrically connected to the moving component; a first motor, disposed on the moving component and electrically connected to the control element; a first rotating rod, connected to the first motor; a second motor, disposed on the moving component and electrically connected to the control element; a second rotating rod, connected to the second motor; and a telescopic component, disposed on the moving component corresponding to the first rotating rod and the second rotating rod, electrically connected to the control element, and movable between a first position and a second position; wherein, the moving component drives the first rotating rod and the second rotating rod to move to be connected with the microtube components, the first motor drives the first rotating rod to rotate one of the microtube components, and the second motor drives the second rotating rod to rotate another one of the microtube components.

In some embodiments, the moving component drives the first rotating rod and the second rotating rod to move to be connected with a plurality of grooves of the microtube components, a mechanical shape of each groove is a straight shape, a cross shape, a star shape, an explosion shape, or a polygon, the first motor drives the first rotating rod to rotate one of the microtube components, and the second motor drives the second rotating rod to rotate another one of the microtube components.

In some embodiments, the telescopic component moves from the first position to the second position to make the microtube components be removed from the first rotating rod and the second rotating rod.

In some embodiments, the telescopic component further includes: a driving motor and a telescopic element, and the telescopic element is connected to the driving motor.

In some embodiments, the telescopic element is deposed outside or inside of the first rotating rod and the second rotating rod.

In some embodiments, the telescopic element includes a plurality of sleeve kits, and the sleeve kits are adapted to sheathe outside of the first rotating rod and the second rotating rod.

In some embodiments, the moving component further including: a first moving element, disposed on the workbench; a second moving element, disposed on the first moving element and movable on the first moving element; a third moving element, disposed on the second moving element and movable on the second moving element; and a sliding block, disposed on the third moving element and movable on the third moving element.

In some embodiments, a moving direction of the second moving element, a moving direction of the third moving element, and a moving direction of the sliding block are perpendicular to one another.

The present disclosure provides a nucleic acid extraction system including: a moving device; and a centrifuge device, disposed corresponding to the moving device and electrically connected to the moving device, and including: a main body; a rotary motor, disposed on the main body; a rotating disk, axially connected to the rotary motor; and a microtube rack, pivotally connected to the rotating disk.

The present disclosure provides a control method of a moving device, applied in a nucleic acid extraction system, the moving device cooperated with a plurality of microtube components, and the control method including: driving, by a moving component of the moving device, a first rotating rod, and a second rotating rod to move to be connected with the microtube components; driving, by a first motor of the moving device, the first rotating rod to rotate one of the microtube components in a first rotation direction, and driving, by a second motor of the moving device, the second rotating rod to rotate another one of the microtube components in the first rotation direction; driving, by the moving device, the first rotating rod and the second rotating rod to move in a vertical direction; and driving, by the first motor, the first rotating rod to rotate one of the microtube components in a second rotation direction, and driving, by the second motor, the second rotating rod to rotate another one of the microtube components in the second rotation direction.

In some embodiments, the control method further including: driving, by the moving component, the first rotating rod and the second rotating rod to take a plurality of sealing screw covers and a plurality of sample tubes of the microtube components from a microtube rack.

In some embodiments, the control method further including: moving a telescopic component from a first position to a second position to make the microtube components be removed from the first rotating rod and the second rotating rod.

In some embodiments, the control method further including: embedding a first protrusion structure of the microtube component in a matching structure of the microtube component; and tightening or loosening, by the first rotating rod and the second rotating rod, a plurality of sealing screw covers of the microtube component.

In summary, the moving device and control method thereof for a nucleic acid extraction system of the present disclosure may let the user only need to place the container containing the buffer solution on the buffer tube rack, put the sample into the sample tube of the microtube component, and then place the sample tube on the sample rack to perform the processes, such as absorbing the buffer solution, discharging the buffer solution to the sample tube which loads the sample, tightening the sealing screw cover, taking the microtube component, moving the microtube component, removing the microtube component, making the microtube component perform the circular motion, etc., through the moving device and control method thereof. In other words, the moving device and control method thereof of the present disclosure may convert the cumbersome, repetitive, and boring processes in the related-art, such as manually tightening the sealing screw cover, taking the microtube component, moving the microtube component, and removing the microtube component, making the microtube component perform the circular motion, etc., into automation to save user's labor. Because the moving device may batch a plurality of the microtube components, the disclosure is more efficient than the manual process of the related-art.

### BRIEF DESCRIPTION OF THE DRAWINGS

FIG. 1 is a three-dimensional diagram of the moving device in accordance with an embodiment of the present disclosure.

FIG. 2 is a three-dimensional diagram of the microtube component in accordance with the present disclosure.

FIG. 3A and FIG. 3B are schematic diagrams of the operation of the telescopic component in accordance with the present disclosure.

FIG. 4 is a three-dimensional diagram of the nucleic acid extraction system in accordance with an embodiment of the present disclosure.

FIG. 5 is a cross-section diagram of the centrifuge device of the nucleic acid extraction system in accordance with the present disclosure.

FIG. 6 is a three-dimensional diagram of the microtube rack of the nucleic acid extraction system in accordance with the present disclosure.

FIG. 7 is a flowchart of the control method of the moving device in accordance with the present disclosure.

FIG. 8A to FIG. 8D are schematic diagrams of the operation of the nucleic acid extraction system in accordance with the present disclosure.

### DETAILED DESCRIPTION

The technical contents of this disclosure will become apparent with the detailed description of embodiments accompanied with the illustration of related drawings as follows. It is intended that the embodiments and drawings disclosed herein are to be considered illustrative rather than restrictive.

As used in the present disclosure, terms such as “first”, “second”, “third”, “fourth”, and “fifth” are employed to describe various elements, components, regions, layers, and/or parts. These terms should not be construed as limitations on the mentioned elements, components, regions, layers, and/or parts. Instead, they are used merely for distinguishing one element, component, region, layer, or part from another. Unless explicitly indicated in the context, the usage of terms such as “first”, “second”, “third”, “fourth”, and “fifth” does not imply any specific sequence or order.

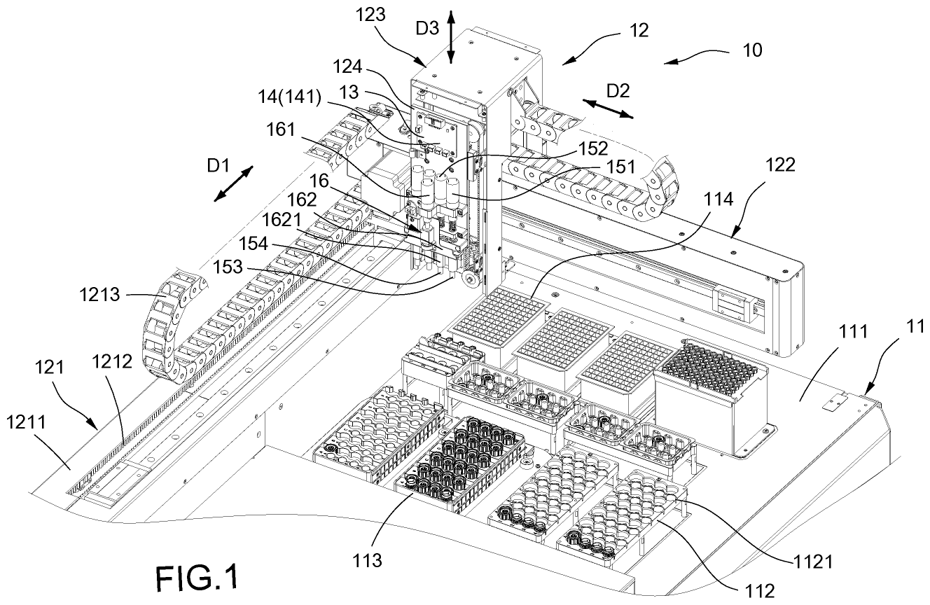

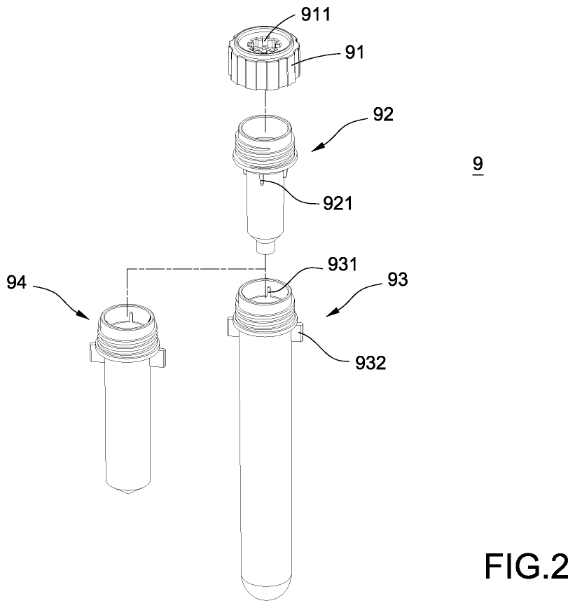

FIG. 1 is a three-dimensional diagram of the moving device 10 in accordance with an embodiment of the present disclosure. FIG. 2 is a three-dimensional diagram of the microtube component 9 in accordance with the present disclosure. Please refer to FIG. 1 and FIG. 2, the moving device 10 of the embodiment is cooperated with the microtube components 9. The moving device 10 of the embodiment includes a workbench 11, a moving component 12, a control element 13, a first motor 151, a first rotating rod 153, a second motor 152, a second rotating rod 154, and a telescopic component 16.

The workbench 11, for example, may include the countertop 111. In some embodiments, the workbench 11, for example, may further include the storage rack 112, the sample rack 113, and the buffer tube rack 114 disposed on the countertop 111. The storage rack 112 is used to, for example, accommodate the sample tube 94 which is loaded with nucleic acid. The sample rack 113 is used to, for example, accommodate the sample tube 94 which is loaded with the sample. The buffer tube rack 114 is used to, for example, accommodate the container that is loaded with the buffer solution.

The moving component 12 is disposed on the workbench 11. In the embodiment, the moving component 12 may include, for example, the first moving element 121, the second moving element 122, the third moving element 123, and the sliding block 124.

The first moving element 121 is disposed on the countertop 111 of the workbench 11. The first moving element 121, for example, may include the linear track body 1211, the conveyor 1212, and the track chain 1213, here is not intended to be limiting. The track chain 1213 is used for protecting and organizing wires to prevent them from falling off or getting tangled. In some embodiments, the first moving element 121 may also be structured by cooperating with the linear track body with a servo motor (not shown in figures) and a threaded pipe (not shown in figures). The servo motor is disposed on the linear track body, and the threaded pipe is axially connected to the servo motor and the linear track body.

The second moving element 122 is disposed on the first moving element 121 and movable on the first moving element 121. The structure of the second moving element 122 is similar to that of the first moving element 121, here is omitted for brevity. When the conveyor 1212 of the first moving element 121 moves, the conveyor 1212 may drive the second moving element 122 to move along the first direction D1 (for example, the left and right direction in FIG. 1) on the linear track body of the first moving element 121.

The third moving element 123 is disposed on the second moving element 122 and is movable on the second moving element 122. The structure of the third moving element 123 is similar to that of the first moving element 121, here is omitted for brevity. When the conveyor of the second moving element 122 moves, the conveyor may drive the third moving element 123 to move along the second direction D2 (for example, the front and back direction in FIG. 1) on the linear track body of the second moving element 122.

The sliding block 124 is disposed on the third moving element 123 and movable on the third moving element 123. When the conveyor of the third moving element 123 moves, the conveyor may drive the sliding block 124 to move along the third direction D3 (for example, the up-down direction in FIG. 1) on the linear track body of the third moving element 123.

As a result, the sliding block 124 may be driven in three-dimensional space by the first moving element 121, the second moving element 122, and the third moving element 123. In some embodiment, the moving direction of the second moving element 122, the moving direction of the third moving element 123, and the moving direction of the sliding block 124 are perpendicular to one another.

The control element 13 is disposed on the moving component 12 and electrically connected to the moving component 12. The control element 13 may include, for example, a sensor integrated circuit, a micro control unit (MCU), a microprocessor unit (MPU), a central process unit, (CPU), an application specific integrated circuit (ASIC), a digital signal processor (DSP), a graphics processing unit (GPU), a field programmable gate array (FPGA), a system on chip (SoC), or other applicable chips, here is not intended to be limiting.

The first motor 151 is disposed on the moving component 12 and electrically connected to the control element 13. In this embodiment, the first motor 151 is disposed on the sliding block 124 and the rotation axis of the first motor 151 is perpendicular to the countertop 111. In some embodiments, the first motor 151 is a servo motor or a stepper motor, here is not intended to be limiting.

The first rotating rod 153 is connected to the first motor 151. In the embodiment, the first rotating rod 153 is axially connected to the first motor 151. The first rotating rod 153 rotates along the rotation axis of the first motor 151. One end of the first rotating rod 153 that contacts the microtube component 9 may be made of elastic material. A mechanical shape of the elastic material may be a straight shape, a cross shape, a star shape, an explosion shape, or a polygon.

The mechanical shape of the elastic material is arranged corresponding to a mechanical shape of a groove 911 on the sealing screw cover 91 of the microtube component 9. The mechanical shape of the groove 911 may be a straight shape, a cross shape, a star shape, an explosion shape, or a polygon. When the moving component 12 drives the first rotating rod 153 to move to be connected with the sealing screw cover 91 of the microtube components 9 by matching the mechanical shape of the elastic material of one end of the first rotating rod 153 and the mechanical shape of the groove 911 on the sealing screw cover 91 of the microtube component 9, the mechanical interference may occur to cause the sealing screw cover 91 to be driven by the first rotating rod 153.

The second motor 152 is disposed on the moving component 12 and electrically connected to the control element 13. In the embodiment, the second motor 152 is disposed on the sliding block 124 and the rotation axis of the second motor 152 is perpendicular to the countertop 111. The second motor 152 is disposed side by side with the first motor 151. The second motor 152 is a servo motor or stepper motor, here is not intended to be limiting.

The second rotating rod 154 is connected to the second motor 152. In the embodiment, the second rotating rod 154 is axially connected to the second motor 152. The second rotating rod 154 rotates along the rotation axis of the second motor 152. The second rotating rod 154 is similar to the first rotating rod 153. One end of the second rotating rod 154 that contacts the microtube component 9 may be made of elastic material. A mechanical shape of the elastic material may be a straight shape, a cross shape, a star shape, an explosion shape, or a polygon. The mechanical shape of the elastic material is arranged corresponding to a mechanical shape of a groove 911 on the sealing screw cover 91 of the microtube component 9. The mechanical shape of the groove 911 may be a straight shape, a cross shape, a star shape, an explosion shape, or a polygon. When the moving component 12 drives the second rotating rod 154 to move to be connected with the sealing screw cover 91 of the microtube components 9 by matching the mechanical shape of the elastic material of one end of the second rotating rod 154 and the mechanical shape of the groove 911 on the sealing screw cover 91 of the microtube component 9, the mechanical interference may occur to cause the sealing screw cover 91 to be driven by the second rotating rod 154.

In some embodiments, the moving device 10 may also include a third motor and a third rotating rod. The third motor is disposed on the moving component 12 and electrically connected to the control element 13. The third rotating rod is connected to the third motor. The moving device 10 may also include a fourth motor and a fourth rotating rod, here is omitted for brevity.

In some embodiments, the microtube component 9 cooperated with the moving device 10 may include, for example, the sealing screw cover 91, the column tube 92, the collection tube 93, and the sample tube 94. It is worth mentioning that the moving device 10 may be cooperated with a plurality of microtube components 9. For example, if the moving device 10 includes two rotating rods, then the moving device 10 may be cooperated with two of the microtube components 9. If the moving device 10 includes four rotating rods, then the moving device 10 may be cooperated with four of the microtube components 9, and so on. The column tube 92 includes the first protrusion structure 921, the collection tube 93 includes a matching structure 931 and the second protrusion structure 932. The matching structure 931 is a concave structure, and the matching structure 931 is arranged corresponding to the first protrusion structure 921. Therefore, when the column tube 92 is placed in the storage space of the collection tube 93, the first protrusion structure 921 may be embedded in the matching structure 931. The sample tube 94 is similar in structure to the collection tube 93, here is omitted for brevity. In some embodiments, the capacity of the column tube 92 is less than that of the sample tube 94. The capacity of the sample tube 94 is less than that of the collection tube 93.

The storage rack 112 of the workbench 11 may include the first groove structure 1121 with the number and shape corresponding to the number and shape of the second protrusion structure 932. Therefore, when the collection tube 93 is placed in the storage space of the storage rack 112, the second protrusion structure 932 may be embedded in the first groove structure 1121. Therefore, the side wall of the second protrusion structure 932 of the collection tube 93 abuts against the side wall of the first groove structure 1121 of the storage rack 112 to make the collection tube 93 be not able to rotate. The sample rack 113 is similar in structure to the storage rack 112, here is omitted for brevity.

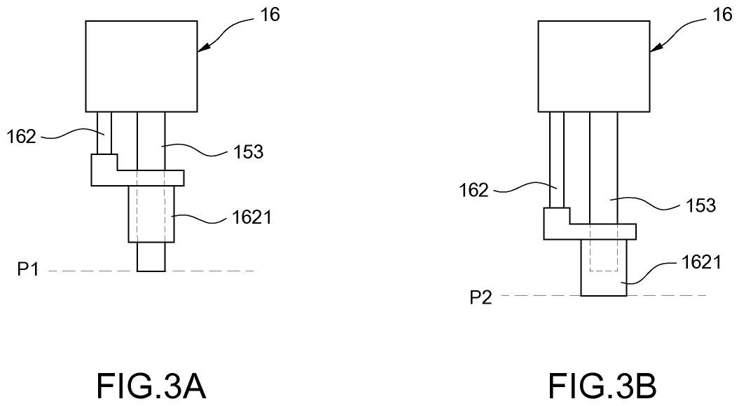

FIG. 3A and FIG. 3B are schematic diagrams of the operation of the telescopic component 16 in accordance with the present disclosure. Please refer to FIG. 1, FIG. 2, FIG. 3A and FIG. 3B, the telescopic component 16 is disposed on the moving component 12 corresponding to the first rotating rod 153 and the second rotating rod 154, electrically connected to the control element 13, and movable between a first position P1 and a second position P2.

In the embodiment, the telescopic component 16 is disposed on the sliding block 124. The telescopic component 16 further includes a driving motor 161 and a telescopic element 162. The telescopic element 162 is connected to the driving motor 161. The driving motor 161 is electrically connected to the control element 13. The telescopic element 162 may be deposed outside or inside of the first rotating rod 153 and the second rotating rod 154. In the embodiment, the telescopic element 162 includes a sleeve kit 1621, and the sleeve kit 1621 is adapted to sheathe outside of the first rotating rod 153 and the second rotating rod 154. The telescopic element 162 is not in contact with the first rotating rod 153 and the second rotating rod 154.

The telescopic component 16 moves from the first position P1 to the second position P2 to make the microtube components 9 be removed from the first rotating rod 153 and the second rotating rod 154. When the driving motor 161 rotates, the sleeve kit 1621 is driven to move from the first position P1 to the second position P2. At this time, the sleeve kit 1621 protrudes from the first rotating rod 153 and the second rotating rod 154 to make the microtube component 9, which is driven by the first rotating rod 153 and the second rotating rod 154, be removed. In other embodiments, the sleeve kit 1621 may also be sheathed inside of the first rotating rod 153 and the second rotating rod 154.

In some embodiments, the moving device 10 may further include the storage element 14 (as shown in FIG. 1) and is electrically connected to the control element 13. The storage element 14 for example includes the predetermined operating commands 141. The control element 13 controls the moving device 10 according to the predetermined operating commands 141, such as controlling the moving component 12 to drive the first rotating rod 153 and the second rotating rod 154 to move to be connected with the microtube components 9, the first motor 151 drives the first rotating rod 153 to rotate one of the microtube components 9, and the second motor 152 drives the second rotating rod 154 to rotate another one of the microtube components 9.

Similarly, the control element 13, for example, controls the first moving element 121, the second moving element 122, and the third moving element 123 to make the sliding block 124 move in three-dimensional space by the first moving element 121, the second moving element 122, and the third moving element 123. Similarly, the control element 13, for example, controls the first motor 151 and the second motor 152 to drive the first rotating rod 153 and the second rotating rod 154. Similarly, the control element 13, for example, controls the driving motor 161 to drive the sleeve kit 1621 to move from the first position P1 to the second position P2. Here is not intended to be limiting.

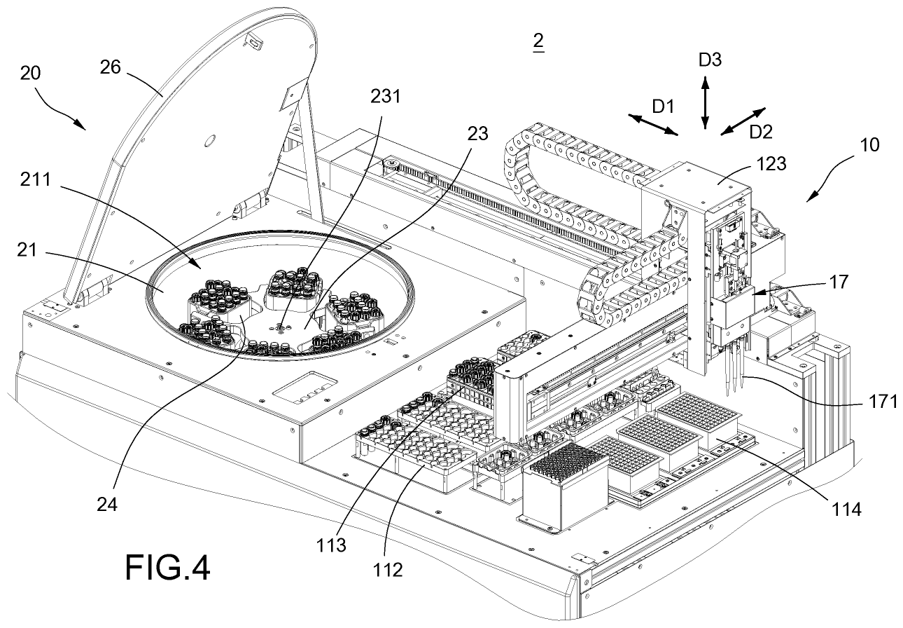

FIG. 4 is a three-dimensional diagram of the nucleic acid extraction system 2 in accordance with an embodiment of the present disclosure. Please refer to FIG. 4, in some embodiments, the moving device 10 may further include the pipetting components 17 disposed on the third moving element 123. The pipetting components 17 may include, for example, a straw 171, which may suck and discharge liquid.

In summary, the moving device 10 of the embodiment cooperated with the microtube component 9 may let the user only need to place the container containing the buffer solution on the buffer tube rack 114, put the sample into the sample tube 94 of the microtube component 9, and then place the sample tube 94 on the sample rack 113 to perform the processes, such as absorbing the buffer solution, discharging the buffer solution to the sample tube 94 which loads the sample, tightening the sealing screw cover 91, taking the microtube component 9, moving the microtube component 9, removing the microtube component 9, etc., through the first motor 151, the first rotating rod 153, the second motor 152, the second rotating rod 154, the telescopic component 16, the pipetting components 17, and other components, of the moving component 12 of the moving device 10. Since the moving device 10 of the embodiment may include equal to or more than two rotary motors 22 and equal to or more than two rotary rods, the moving device 10 may process equal to or more than two microtube components 9 at a time. In some embodiments, the moving device 10 of the embodiment may process four microtube components 9 at a time through the third motor, the third rotating rod, the fourth motor, and the fourth rotating rod. As a result, the efficiency is greatly increased. In other words, the moving device 10 of the embodiment may convert the cumbersome, repetitive, and boring processes in the related-art, such as manually tightening the sealing screw cover 91, taking the microtube component 9, moving the microtube component 9, and removing the microtube component 9, etc., into automation to save user's labor. Because the moving device 10 may batch a plurality of the microtube components 9, the embodiment is more efficient than the manual process of the related-art. In addition, compared with the related-art automated machines on the market, a plurality of mold tubes, such as the microtube components 9, may not be operated simultaneously due to technical bottlenecks. The moving device 10 of the embodiment may process a plurality of microtube components 9 in batches. As a result, the efficiency is greatly increased.

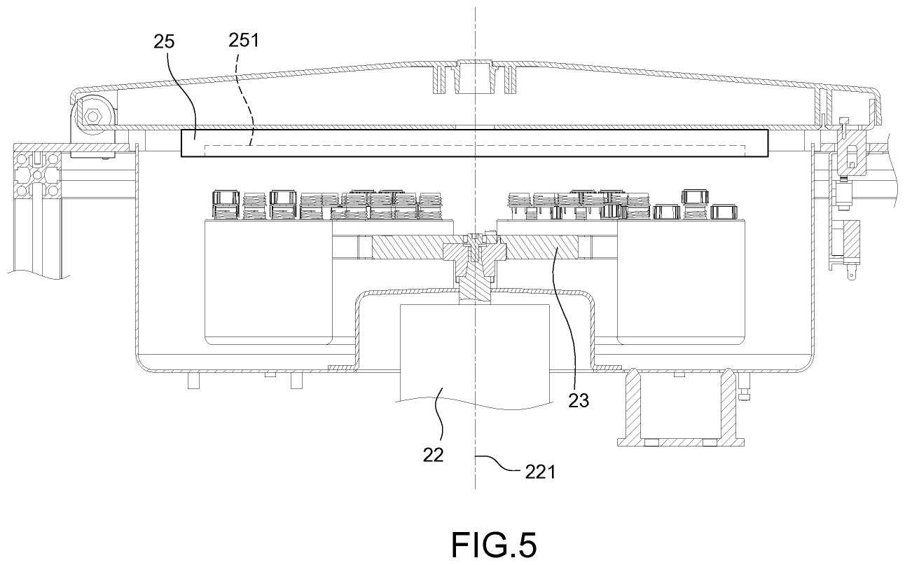

FIG. 4 is a three-dimensional diagram of the nucleic acid extraction system 2 in accordance with an embodiment of the present disclosure. FIG. 5 is a cross-section diagram of the centrifuge device 20 of the nucleic acid extraction system 2 in accordance with the present disclosure. Please refer to FIG. 4 and FIG. 5, the nucleic acid extraction system 2 of this embodiment may be cooperated with the moving device 10 of FIG. 1, here is not intended to be limiting. The moving device may also adopt different the moving devices other than the moving device 10. The nucleic acid extraction system 2 of the embodiment includes the moving device 10 and the centrifuge device 20. The moving device 10 may, for example, adopt the moving device 10 as shown in FIG. 1, 3A, 3B, here is omitted for brevity. Additionally, in some embodiments, the moving device 10 may further include the pipetting components 17 disposed on the third moving element 123. The pipetting components 17 may include, for example, a straw 171, which may suck and discharge liquid.

The centrifuge device 20 is used for extracting nucleic acids from biological samples. The centrifuge device 20 may include the main body 21, the rotary motor 22, the rotating disk 23, and the microtube rack 24.

The main body 21 in the embodiment, for example, may include the chamber 211. The rotary motor 22, the rotating disk 23, and the microtube rack 24 are disposed in a chamber 211.

The rotary motor 22 is disposed on the main body 21. The rotary motor 22 may be completely disposed inside the chamber 211, or only the rotary motor axis 221 of the rotary motor 22 may be disposed inside the chamber 211.

The rotating disk 23 includes a rotating disk axis 231 which is axially connected to the rotary motor 22. In the embodiment, when the rotary motor 22 operates, the rotary motor 22 may drive the rotating disk 23 to rotate horizontally with the rotary motor axis 221 as the center in the chamber 211.

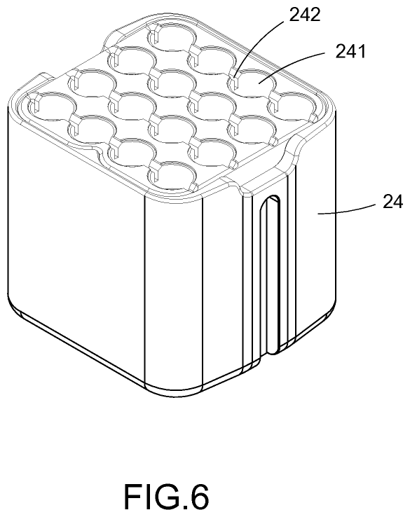

FIG. 6 is a three-dimensional diagram of the microtube rack 24 of the nucleic acid extraction system in accordance with the present disclosure. Please refer to FIG. 2, FIG. 4, and FIG. 6, the microtube rack 24, pivotally connected to the rotating disk 23. In this embodiment, the microtube rack 24 includes the hole 241 and the second groove structure 242. The number and shape of the second groove structure 242 is arranged corresponding to the second protrusion structure 932 of the collection tube 93. Therefore, when the collection tube 93 is placed in the storage space of the hole 241, the second protrusion structure 932 may be embedded in the second groove structure 242. Therefore, the side wall of the second protrusion structure 932 of the collection tube 93 abuts against the side wall of the second groove structure 242 of the microtube rack 24 to make the collection tube 93 be not able to rotate.

Please refer to FIG. 2, FIG. 4, and FIG. 5, when the rotary motor 22 drives the rotating disk 23 to rotate, the microtube rack 24 and the microtube component 9 disposed on the microtube rack 24 are driven by the rotating disk 23. As a result, the microtube rack 24 and the microtube component 9 perform circular motion inside the chamber 211 along the rotary motor axis 221 as the center.

In some embodiments, the centrifuge device 20 may further include the cover body 26 corresponding to the main body 21. Before the rotary motor 22 starts, the cover body 26 may be closed on the chamber 211 to prevent the user's hand from accidentally inserting into the chamber 211 after the rotary motor 22 starts, which may hurt the user's hand due to the collision with the rotating disk 23, the microtube rack 24, or the microtube component 9.

In some embodiments, the centrifuge device 20 may include the heating module 25 disposed on the main body 21. The heating module 25 is disposed inside the main body 21 and is used for heating the chamber 211 to the operating ambient temperature. In some embodiments, the heating module 25 may include the heating plate 251 and be attached to the inner surface of the cover body 26. It should be noted that the shape and disposed position of the heating module 25 are not limited to this. The heating module 25 may also be a heating rod or other device that includes a heating function, and the heater may also be disposed at other positions inside the main body 21 without mechanical interference with the rotary motor 22, the rotating disk 23, and the microtube rack 24.

In summary, the nucleic acid extraction system 2 of the embodiment may be cooperated with the microtube component 9 may let the user only need to place the container containing the buffer solution on the buffer tube rack 114, put the sample into the sample tube 94 of the microtube component 9, and then place the sample tube 94 on the sample rack 113 to perform the processes, such as absorbing the buffer solution, discharging the buffer solution to the sample tube 94 which loads the sample, tightening the sealing screw cover 91, taking the microtube component 9, moving the microtube component 9, removing the microtube component 9, making the microtube component 9 perform the circular motion, etc., through the first motor 151, the first rotating rod 153, the second motor 152, the second rotating rod 154, the telescopic component 16, the pipetting components 17, and other components, of the moving component 12 of the moving device 10. Since the moving device 10 may include equal to or more than two rotary motors 22 and equal to or more than two rotary rods, the moving device 10 may process equal to or more than two microtube components 9 at a time. In some embodiments, the moving device 10 may process four microtube components 9 at a time through the third motor, the third rotating rod, the fourth motor, and the fourth rotating rod. As a result, the efficiency is greatly increased. In other words, the nucleic acid extraction system 2 of the embodiment may convert the cumbersome, repetitive, and boring processes in the related-art, such as manually tightening the sealing screw cover 91, taking the microtube component 9, moving the microtube component 9, and removing the microtube component 9, making the microtube component 9 perform the circular motion, etc., into automation to save user's labor. Because the moving device 10 may batch a plurality of the microtube components 9, the embodiment is more efficient than the manual process of the related-art. In addition, compared with the related-art automated machines on the market, a plurality of mold tubes, such as the microtube components 9, may not be operated simultaneously due to technical bottlenecks. The nucleic acid extraction system 2 of the embodiment may process a plurality of microtube components 9 in batches. As a result, the efficiency is greatly increased.

In addition, the centrifuge device 20 of the nucleic acid extraction system 2 of this embodiment may extract nucleic acid from the sample in the microtube component 9.

Furthermore, the heating module 25 may be used to directly heat and dry the liquid on the membrane filter of the centrifuge tube after the centrifuge device 20 completes the centrifugation. The embodiment does not require additional high-speed operation or moving to other heating equipment. As a result, process time and equipment costs may be reduced.

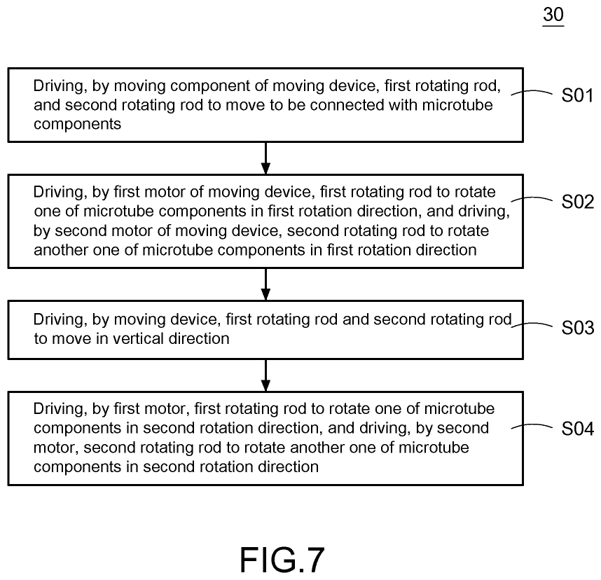

FIG. 7 is a flowchart of the control method of the moving device in accordance with the present disclosure. The control method of the moving device may adopt the moving device 10 as shown in FIG. 1, here is not intended to be limiting. As shown in FIG. 7, the step S01 is driving, by a moving component of the moving device, a first rotating rod, and a second rotating rod to move to be connected with the microtube components. The step S02 is driving, by the first motor of the moving device, the first rotating rod to rotate one of the microtube components in a first rotation direction, and driving, by a second motor of the moving device, the second rotating rod to rotate another one of the microtube components in the first rotation direction. The step S03 is driving, by the moving device, the first rotating rod and the second rotating rod to move in a vertical direction. The step S04 is driving, by the first motor, the first rotating rod to rotate one of the microtube components in a second rotation direction, and driving, by the second motor, the second rotating rod to rotate another one of the microtube components in the second rotation direction.

As shown in FIG. 1, FIG. 2, FIG. 4, FIG. 5, and FIG. 7, in some embodiments, before step S01, the control element 13 of the moving device 10 may control the pipetting components 17 first absorbs the buffer solution, and then discharges the buffer solution into the sample tube 94 on the sample rack 113. In some embodiments, the sample rack 113 may be shaken and heated, here is not intended to be limiting. The control element 13 then controls the pipetting components 17 to absorb the sample and buffer solution in the sample tube 94. Then the control element 13 controls the pipetting components 17 to discharge the sample and buffer solution on the column tube 92 inside the collection tube 93 inside the microtube rack 24. In other words, the column tube 92 is sheathed inside the collection tube 93. The control element 13 controls the cover body 26 of the centrifuge device 20 covering the main body 21. As a result, the sample and buffer solution are mixed and placed in the centrifuge device 20 in preparation for centrifugation.

The control element 13 controls the rotary motor 22 of the centrifuge device 20 driving the rotating disk 23 of the centrifuge device 20 to cause the microtube component 9 to perform circular motion. After completing the centrifugation operation, the control element 13 controls the centrifuge device 20 to stop the rotary motor 22. The control element 13 controls the cover body 26 of the centrifuge device 20 to open. As a result, a centrifugation operation is performed to extract nucleic acids.

In some embodiments, if the centrifugation operation is only performed once, impurities other than nucleic acids may still remain on the column tube 92, so the above steps of adding buffer solution and centrifugation may be repeated two to three times to cause the purer nucleic acids to leave on the column tube 92, while impurities are loaded into the collection tube 93. As a result, a plurality of centrifugation operations are performed to extract nucleic acids.

The control element 13 of the moving device 10 may control the first moving element 121, the second moving element 122, and the third moving element 123 of the moving component 12 to drive the sliding block 124, the first rotating rod 153, and the second rotating rod 154. The control element 13 controls the rotation of the first motor 151 and the second motor 152 to drive the first rotating rod 153 and the second rotating rod 154 rotate together to make the first rotating rod 153 and the second rotating rod 154 and the sealing screw cover 91 be connected to cause mechanical interference. As a result, the moving device 10 grabbed the sealing screw cover 91.

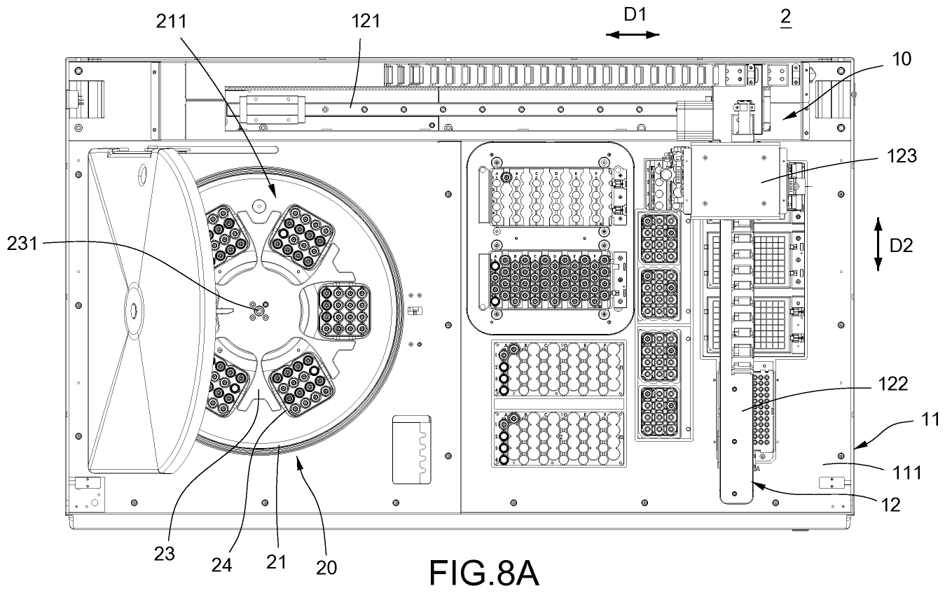

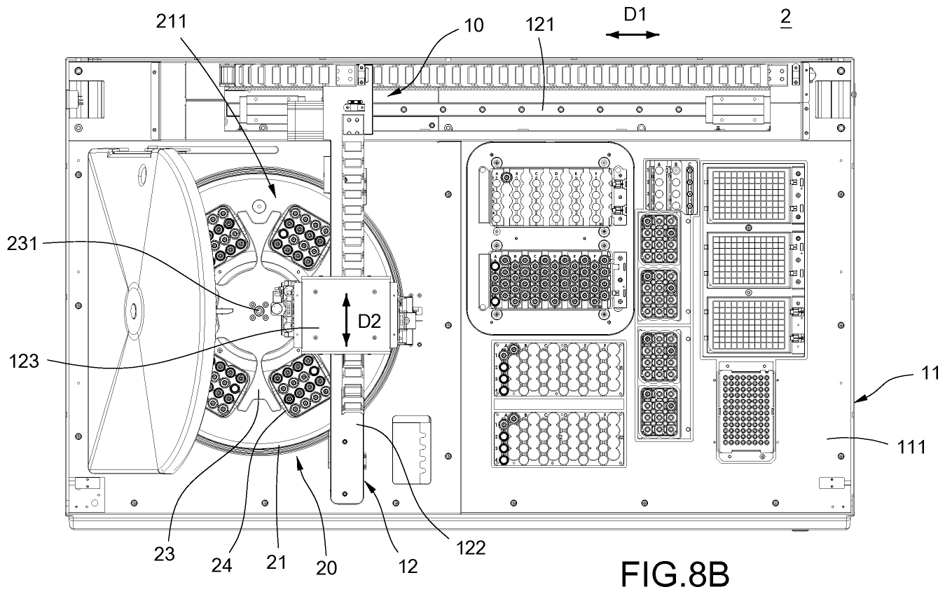

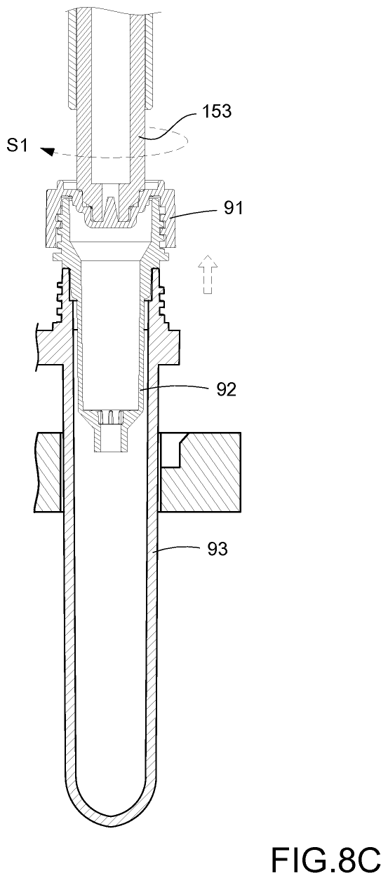

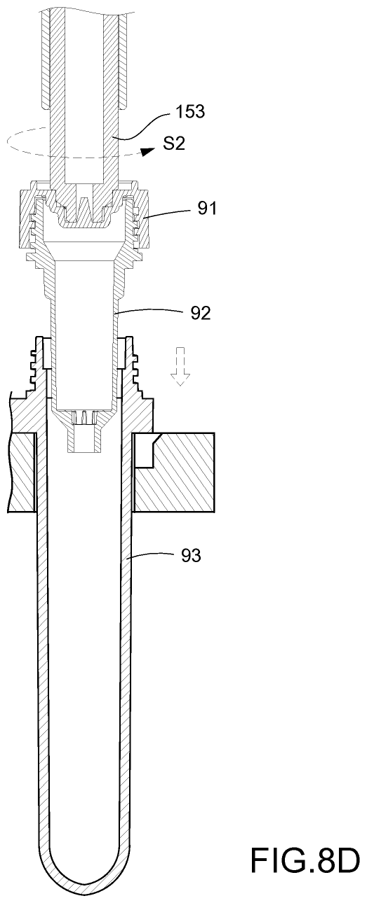

FIG. 8A to FIG. 8D are schematic diagrams of the operation of the nucleic acid extraction system 2 in accordance with the present disclosure. As shown in FIG. 1, FIG. 2, FIG. 7, FIG. 8A, and FIG. 8B, the moving component 12 drives the first rotating rod 153 and the second rotating rod 154 onto the microtube rack 24 of the centrifuge device 20. In other words, the control element 13 may control the moving component 12 to make the sealing screw covers 91 which are fixed on the first rotating rod 153, and the second rotating rod 154 move onto the column tube 92 which completes the centrifugation operation. As a result, the moving device 10 moves onto the centrifuge device 20.

In step S01, the moving component 12 of the moving device 10 drives a first rotating rod 153, and a second rotating rod 154 to move to be connected with the microtube components 9. In the embodiment, the moving component 12 of the moving device 10 may drive the first rotating rod 153, the second rotating rod 154, and the sealing screw cover 91 to the centrifuge device 20. As a result, the column tubes 92 of the microtube components 9 are connected with the sealing screw cover 91.

As shown in FIG. 1, FIG. 2, FIG. 7 and FIG. 8 C, in step S02, the first motor 151 of the moving device 10 drives the first rotating rod 153 to rotate one of the microtube components 9 in a first rotation direction S1, and the second motor 152 of the moving device 10 drives the second rotating rod 154 to rotate another one of the microtube components 9 in the first rotation direction S1. In the embodiment, the first rotation direction S1 is the direction in which the sealing screw cover 91 is tightened to the column tube 92. When the first motor 151 and the second motor 152 rotate in the first rotation direction S1, the first rotating rod 153 and the second rotating rod 154 are driven to rotate in the first rotation direction S1, and the sealing screw cover 91 is driven to rotate in the first rotation direction S1, the column tube 92 does not rotate. As a result, the sealing screw cover 91 may be tightened on the column tube 92.

It is worth mentioning that the microtube rack 24 includes the hole 241 and the second groove structure 242. The number and shape of the second groove structure 242 is arranged corresponding to the second protrusion structure 932 of the collection tube 93. Therefore, when the collection tube 93 is placed in the storage space of the hole 241, the second protrusion structure 932 is embedded in the second groove structure 242. When embedding the first protrusion structure 921 of the microtube component 9 in the matching structure 931 of the microtube component 9, the first protrusion structure 921 is embedded in the matching structure 931, and the second protrusion structure 932 is embedded in the second groove structure 242. In this situation, when the sealing screw covers 91 is rotated by the first rotating rod 153 and the second rotating rod 154, the column tubes 92 and the collection tubes 93 do not rotate. As a result, the sealing screw covers 91 of the microtube components 9 is tightened by the first rotating rod 153 and the second rotating rod 154. In some embodiment, the sealing screw covers 91 of the microtube components 9 is loosened by the first rotating rod 153 and the second rotating rod 154 through a similar method.

In step S03, the moving device 10 drives the first rotating rod 153 and the second rotating rod 154 to move in the vertical direction. In the embodiment, when the first rotating rod 153 and the second rotating rod 154 tighten the sealing screw covers 91 along the first rotation direction S1, the first protrusion structures 921 of the column tubes 92 apply external torque on the collection tubes 93. Because the collection tubes 93 are connected side by side, the collection tubes 93 twist and deform, making the collection tubes 93 and the column tubes 92 above the collection tubes 93 may cause mechanical interference. Therefore, when the first rotating rod 153 and the second rotating rod 154 move in the vertically upward direction, the sealing screw cover 91, the column tube 92, and the collection tube 93 are simultaneously driven to move up.

As shown in FIG. 1, FIG. 2, FIG. 7 and FIG. 8D, in step S04, the first motor 151 drives the first rotating rod 153 to rotate one of the microtube components 9 in a second rotation direction S2, and the second motor 152 drives the second rotating rod 154 to rotate another one of the microtube components 9 in the second rotation direction S2. In the embodiment, the second rotation direction S2 is the direction in which the sealing screw cover 91 is loosened from the column tube 92. The first motor 151 and the second motor 152 rotate in the second rotation direction S2, and the first rotating rod 153 and the second rotating rod 154 are driven to rotate in the second rotation direction S2. As a result, the distortion and deformation of collection tube 93 caused by the external torque caused by the tightening of column tube 92 is released. As a result, the collection tube 93 is freed from the mechanical interference with the column tube 92 above the collection tube 93. As a result, the collection tube 93 is loosened from the column tube 92.

As shown in FIG. 1, FIG. 2, FIG. 4, and FIG. 8D, in some embodiments, the moving device 10 drives the first rotating rod 153, the second rotating rod 154, and the column tube 92 onto the sample tube 94 in the microtube rack 24 of the centrifuge device 20, the first motor 151 and the second motor 152 rotate in the second rotation direction S2 to loosen the column tube 92 and the sealing screw cover 91, so that the column tube 92 is placed on the sample tube 94. Then, the pipetting assembly 17 sucks the buffer solution in the container, such as pure water, and discharged the buffer solution into the column tube 92. Then perform centrifugation operations similar to the above, here is omitted for brevity. As a result, nucleic acids are loaded on the sample tube 94.

The moving device 10 tightens the sealing screw cover 91 on the column tube 92. Then the moving device 10 drives the first rotating rod 153, the second rotating rod 154, and the column tube 92 to the collection tube 93 in the microtube rack 24 of the centrifuge device 20. The first motor 151 and the second motor 152 rotate in the second rotation direction S2 so that the column tube 92 is loosened from the sealing screw cover 91. As a result, the column tube 92 is placed on the collection tube 93. Then, the moving device 10 tightens the sealing screw cover 91 on the sample tube 94. As a result, the sample tube 94 loaded with nucleic acids is driven by the moving device 10.

As shown in FIG. 1, FIG. 2, FIG. 3A, FIG. 3B, FIG. 8A, and FIG. 8B, the moving component 12 drives the first rotating rod 153 and the second rotating rod 154 to take the sealing screw covers 91 of the microtube components 9 and the sample tube 94 from the microtube rack 24. Then the telescopic component 16 moves to above the storage rack 112. The telescopic component 16 moves from the first position P1 to the second position P2 to make the microtube components 9 be removed from the first rotating rod 153 and the second rotating rod 154. In the embodiment, the telescopic component 16 protrudes from the first rotating rod 153 and the second rotating rod 154, so that the sealing screw cover 91 and the sample tube 94 driven by the first rotating rod 153 and the second rotating rod 154 are released to the storage rack 112. As a result, the sample tube 94 loaded with nucleic acid is stored in the storage rack 112.

In summary, the moving device and control method thereof for a nucleic acid extraction system of the present disclosure may let the user only need to place the container containing the buffer solution on the buffer tube rack, put the sample into the sample tube of the microtube component, and then place the sample tube on the sample rack to perform the processes, such as absorbing the buffer solution, discharging the buffer solution to the sample tube which loads the sample, tightening the sealing screw cover, taking the microtube component, moving the microtube component, removing the microtube component, making the microtube component perform the circular motion, etc., through the moving device and control method thereof. In other words, the moving device and control method thereof of the present disclosure may convert the cumbersome, repetitive, and boring processes in the related-art, such as manually tightening the sealing screw cover, taking the microtube component, moving the microtube component, and removing the microtube component, making the microtube component perform the circular motion, etc., into automation to save user's labor. Because the moving device may batch a plurality of the microtube components, the disclosure is more efficient than the manual process of the related-art.

While this disclosure has been described by means of specific embodiments, numerous modifications and variations may be made thereto by those skilled in the art without departing from the scope and spirit of this disclosure set forth in the claims.

What is claimed is:

1. A moving device, applied in a nucleic acid extraction system and cooperated with a plurality of microtube components, the moving device comprising:
a workbench;
a moving component, disposed on the workbench;
a control element, disposed on the moving component and electrically connected to the moving component;
a first motor, disposed on the moving component and electrically connected to the control element;
a first rotating rod, connected to the first motor;
a second motor, disposed on the moving component and electrically connected to the control element;
a second rotating rod, connected to the second motor; and
a telescopic component, disposed on the moving component corresponding to the first rotating rod and the second rotating rod, electrically connected to the control element, and movable between a first position and a second position;
wherein, the moving component drives the first rotating rod and the second rotating rod to move to be connected with the microtube components, the first motor drives the first rotating rod to rotate one of the microtube components, and the second motor drives the second rotating rod to rotate another one of the microtube components.

2. The moving device of claim 1, wherein, the moving component drives the first rotating rod and the second rotating rod to move to be connected with a plurality of grooves of the microtube components, a mechanical shape of each groove is a straight shape, a cross shape, a star shape, an explosion shape, or a polygon, the first motor drives the first rotating rod to rotate one of the microtube components, and the second motor drives the second rotating rod to rotate another one of the microtube components.

3. The moving device of claim 1, wherein, the telescopic component moves from the first position to the second position to make the microtube components be removed from the first rotating rod and the second rotating rod.

4. The moving device of claim 1, wherein, the telescopic component further comprises: a driving motor and a telescopic element, and the telescopic element is connected to the driving motor.

5. The moving device of claim 4, wherein, the telescopic element is deposed outside or inside of the first rotating rod and the second rotating rod.

6. The moving device of claim 5, wherein, the telescopic element comprises a plurality of sleeve kits, and the sleeve kits are adapted to sheathe outside of the first rotating rod and the second rotating rod.

7. The moving device of claim 1, wherein the moving component further comprising:
a first moving element, disposed on the workbench;
a second moving element, disposed on the first moving element and movable on the first moving element;
a third moving element, disposed on the second moving element and movable on the second moving element; and
a sliding block, disposed on the third moving element and movable on the third moving element.

8. The moving device of claim 7, wherein, a moving direction of the second moving element, a moving direction of the third moving element, and a moving direction of the sliding block are perpendicular to one another.

9. A nucleic acid extraction system comprising:
the moving device of claim 1; and
a centrifuge device, disposed corresponding to the moving device and electrically connected to the moving device, and comprising:
a main body;
a rotary motor, disposed on the main body;
a rotating disk, axially connected to the rotary motor; and
a microtube rack, pivotally connected to the rotating disk.

10. A control method of a moving device, applied in a nucleic acid extraction system, the moving device cooperated with a plurality of microtube components, and the control method comprising:
driving, by a moving component of the moving device, a first rotating rod, and a second rotating rod to move to be connected with the microtube components;
driving, by a first motor of the moving device, the first rotating rod to rotate one of the microtube components in a first rotation direction, and driving, by a second motor of the moving device, the second rotating rod to rotate another one of the microtube components in the first rotation direction;
driving, by the moving device, the first rotating rod and the second rotating rod to move in a vertical direction; and
driving, by the first motor, the first rotating rod to rotate one of the microtube components in a second rotation direction, and driving, by the second motor, the second rotating rod to rotate another one of the microtube components in the second rotation direction.

11. The control method of claim 10, further comprising:
driving, by the moving component, the first rotating rod and the second rotating rod to take a plurality of sealing screw covers and a plurality of sample tubes of the microtube components from a microtube rack.

12. The control method of claim 11, further comprising:
moving a telescopic component from a first position to a second position to make the microtube components be removed from the first rotating rod and the second rotating rod.

13. The control method of claim 10, further comprising:
embedding a first protrusion structure of the microtube component in a matching structure of the microtube component; and
tightening or loosening, by the first rotating rod and the second rotating rod, a plurality of sealing screw covers of the microtube component.
